(19) United States
(12) Patent Application Publication
(10) Pub. No.: US 2016/0022479 A1
(43) Pub. Date: Jan. 28, 2016

(54) Externally Heated Thermal Pack

(71) Applicants: Fredrik Ove Haug RINGVOLD, (US); Christian RINGVOLD, (US); NORMEDICAL GROUP AS, Oslo (NO)

(72) Inventors: Christian Ringvold, Skaneateles, NY (US); Fredrik Ove Haug Ringvold, Oslo (NO)

(73) Assignee: NorMedical Group AS, Oslo (NO)

(21) Appl. No.: 14/774,153

(22) PCT Filed: Mar. 13, 2014
(86) PCT No.: PCT/US14/25230

§ 371 Date: Sep. 10, 2015

Related U.S. Application Data
(60) Provisional application No. 61/779,827, filed on Mar. 13, 2013.

(51) Int. Cl. A61F 7/02; C09K 5/10

(52) U.S. Cl. CPC A61F 7/02; C09K 5/10; A61F 2007/0214; A61F 2007/0219; A61F 2007/0222

(57) ABSTRACT

An improved, externally heated thermal pack, has an enclosure or bladder comprised of a skin of a durable flexible film, and has a fill of a blend of propylene glycol (mono- or poly-), water, and a superabsorbent cross-linked polyacrylic salt. The fill is hermetically sealed within the enclosure or bladder. Favorably, the polyacrylic salt and the propylene glycol/water solution are at a ratio in a range of about 21% to 28%. The thermal packs can be in a range of sizes and styles, and exhibit improved stability, better flexibility, smaller risk of failure, reduced risk of scalding or burning the patient, less risk of bacterial contamination and greater service life.

This patent application is a national application filing of International PCT Application PCT/US2014/025230, filed Mar. 13, 2014, and claims priority of Provisional U.S. Patent Application 61/779,827, Mar. 13, 2013, the disclosure of which is incorporated herein.

NorMedical Group AS have invented an improved externally heated thermal pack, available in a range of sizes and styles, with improved stability, better flexibility, less risk of failure, less risk of burning patient, less risk of bacterial contamination and greater service life than currently-available thermal (cloth) packs.

Thermal packs have frequently been used in medical practices, by physical therapists, chiropractors and other practitioners to apply a steady, prolonged heat (up to about 130° F.) to a patient to relax muscles and to prepare for further medical or other therapy or for manipulation.

The current thermal (cloth) packs have several weaknesses. These have to be heated up to 165° F. to kill bacteria, which then leads to risk of burn and requires use of a numerous towels or terry covers. Also, many people are sensitive to latex or to certain plasticizers, and it has been found that the skin material used on the pack envelope can also irritate some patients when the heat pack is in contact with them.

NorMedical Group AS new invented Thermal Pack is more flexible, and can therefore be used in lower temperature. The surface is easy to clean due to the non-latex plastic material (described below) used, as well as the pack does not need to heated up to 165 F to kill bacteria which was required with the older style packs.

The externally heated thermal packs have an outer cover or skin or bladder formed of a supple but tough plastic material, and contain a fill of a blend of a cross-linked polyacrylate salt, monopropylene glycol, and water.

Ideally, the packs are placed in a bath of heated water (controlled at about 131° to 140° F.) for at least 20 minutes to preheat them to 130°. The pre-heated pack is then laid upon the patient's affected body area, with one layer or thickness of toweling in between the patient and the pack in order to provide heat at approximately 120° F. to the patient's tissue. Depending upon the patient, optimal therapeutic temperature is considered to be 112 F to 120 F. After the pack has remained in place for the required amount of time (the pack will hold its temperature for an hour or more, with decreasing temperature changes of less than 3 degrees per 5 minutes) the pack can be removed and placed back into the water bath. A disinfecting step can be added if there has been contact with the patient's skin

In a preferred embodiment, the preferred cross-linked polymer salt is a polyacrylic acid sodium salt, cross-linked, provided in the form of white granules. This is blended with a mixture of monopropylene glycol and water. Preferably the monopropylene glycol is mixed together with water that has been preheated to a temperature of 32-35° C. or 90-95° F. The glycol and water are blended in a ratio of 32 parts by volume glycol to 68 parts by volume water for a total of 100 parts by volume.

In one favorable implementation, the mixture of the superabsorbent cross linked polyacrylic salt with the fluid is at a ratio of 100 parts polyacrylic to about 414 parts glycol/water solution, or a ratio of about 24.25% poly to glycol. This specific ratio has been found to give peak performance, although blends with good performance can have a ratio of poly to glycol of perhaps 23% to 27%, and acceptable performance at 21% to 28%.

For the skin or bladder, the preferred material is a soft PVC film with a thickness of 0.30 mm to 0.50 mm. This material retains strength and flexibility even after long use, and is not prone to developing pinhole leaks or cracks along its seams. The material can also be sealed at the fill nipple by means of an RF heating technique.

The packs can be made in a variety of sizes depending on the patient and the area where heat therapy is needed.

The packs provide stable, non-excessive temperature for thermal treatments, for either dry or wet heat (wet heat can be created by soaking a towel in water before applying to the patient). The packs are hygienic, and can be easily disinfected and hydro-reheated for many uses over their lifetime.

Because normal toweling can be used between the pack and the patient, there is no need for expensive terry covers of the type that were needed for the older version of the heat pack.

The invention is not to be limited only to the described embodiment, but is to be determined by the following Claims:

1. A thermal pack formed of an enclosure or bladder comprised of a skin of a durable flexible film, and a fill of a blend of propylene glycol (mono or poly), water, and a superabsorbent cross-linked polyacrylic salt, the fill being hermetically sealed within the enclosure or bladder, wherein the propylene glycol and water are blended at about 3 parts propylene glycol to about 7 parts water by volume and in which the cross-linked polyacrylic salt and the propylene glycol/water solution are at a ratio in a range of about 21% to 28%.

2. The thermal pack of claim 1 wherein said ratio is between about 23% to 27%.

3. The thermal pack of claim 1 wherein said ratio is between about 24% to 25%

4. The thermal pack of claim 1 wherein said ratio is substantially 24¼%.

5. The thermal pack of claim 1 wherein the skin of said enclosure or bladder is a PVC film about 0.30 to 0.50 mm in thickness.

6. The thermal pack of claim 1 wherein the propylene glycol and water are blended at about 32 to 68 parts, respectively, by volume.
